(19) United States
(12) Plant Patent Application Publication
(10) Pub. No.: US 2011/0126331 P1
(43) Pub. Date: May 26, 2011

(54) Apple tree named 'Gradirose'

(50) ;

(76) Inventors: Alexandre Grard, Mudaison (FR); Olivier Grard, Mudaison (FR)

(21) Appl. No.: 12/807,458

(22) Filed: Sep. 3, 2010

Related U.S. Application Data
(60) Provisional application No. 61/276,490, filed on Sep. 11, 2009.

(51) Int. Cl. A01H 5/00

(52) U.S. Cl. PLT/161

(57) ABSTRACT

‘Gradirose’ is a new and distinct apple tree notable for its semi-upright plant habit, skin over color, early timing of fruit ripening, and fruit quality.

### CROSS REFERENCE TO RELATED APPLICATIONS

This application claims priority of co-pending U.S. Provisional Application Ser. No. 61/276,490 filed Sep. 11, 2009.

### STATEMENT REGARDING FEDERALLY SPONSORED RESEARCH OR DEVELOPMENT

None

### LATIN NAME OF THE GENUS AND SPECIES OF THE PLANT CLAIMED

Malus domestica (Borkh.)

### VARIETY DENOMINATION

‘Gradirose’

### BACKGROUND OF THE INVENTION

The present invention relates to a new and distinct cultivar of apple tree, botanically known as Malus domestica, and hereinafter referred to by the name ‘Gradirose’.

The new apple variety ‘Gradirose’ originated from an open-pollination in Hérault, France of Malus domestica ‘Christmas Rose’, (not patented), as the female, or seed parent, with an unknown selection of Malus domestica, as the male or pollen, parent. The new Apple tree was discovered and selected by the inventors as a single plant from within the progeny of the stated open-pollination in a controlled environment in Hérault, France in 1991.

Asexual reproduction of the new apple tree by budding and grafting, since 1994 in Hérault, France, has demonstrated that the new variety reproduces true to type with all of the characteristics, as herein described, firmly fixed and retained through successive generations.

### SUMMARY OF THE INVENTION

The following characteristics of the new variety have been repeatedly observed and can be used to distinguish ‘Gradirose’ as a new and distinct cultivar of apple:
1. Semi-upright plant habit.
2. Skin over color.
3. Early timing of fruit ripening.
4. Fruit quality.

### BRIEF DESCRIPTION OF THE PHOTOGRAPHS

FIG. 1 shows the tree of the new variety;

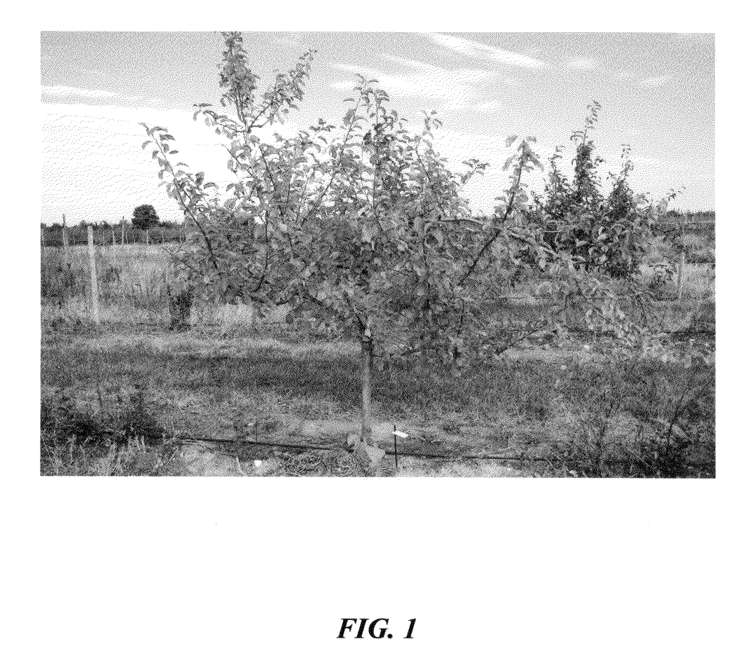

FIG. 2 shows the tree and flower of the new variety;

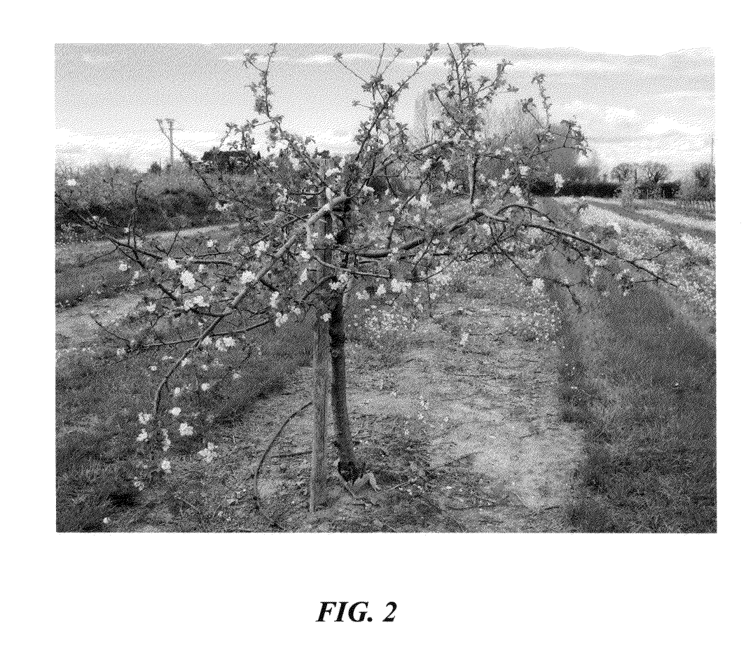

FIG. 3 shows the fruit and leaves of the new variety;

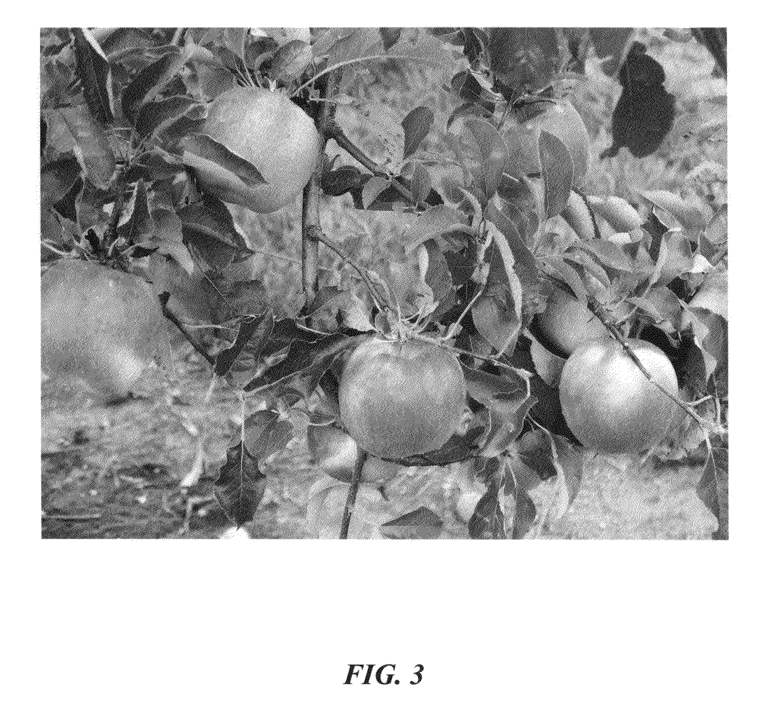

FIG. 4 shows the fruit of the new variety; and

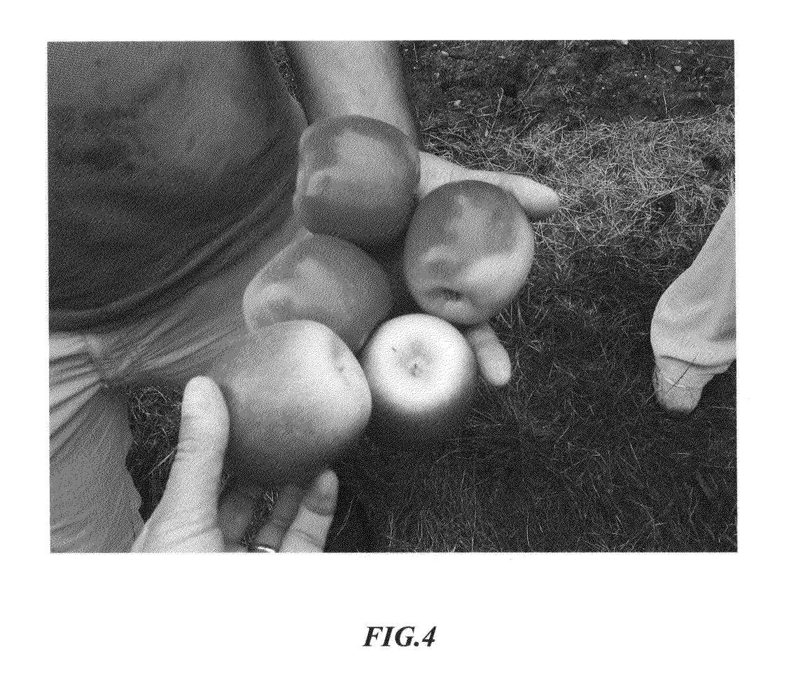

FIG. 5 shows the blossom of the new variety.

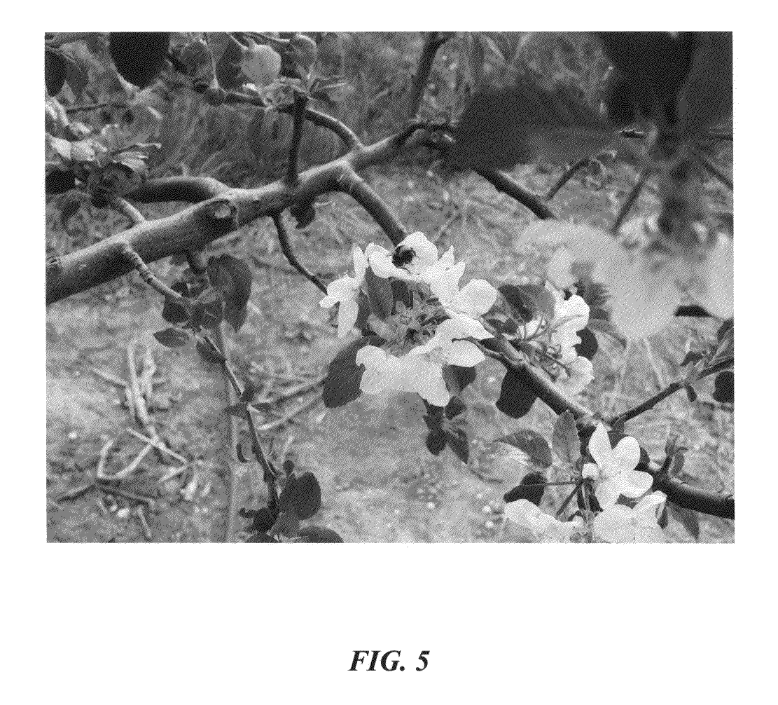

### DETAILED BOTANICAL DESCRIPTION

The following detailed botanical description is based on observations of trees planted in 1995, and described during the 2003 and 2004 growing seasons at Hérault, France. Color descriptions refer to The Royal Horticultural Society Colour Chart (1995). It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Botanical classification: Malus domestica Borkh cultivar Gradirose.
Parentage:
   Female, or seed parent.—Malus domestica ‘Christmas Rose’, not patented.
   Male, or pollen, parent.—Unknown selection of Malus domestica, not patented.
Tree:
   Vigor.—Vigorous.
   Habit.—Semi-Upright.
   Size.—Height: 3.0 m.
   Trunk.—Diameter: 15-20 cm at 30 cm above graft union; bark texture smooth; color brown RHS N200C.
   Branches.—1 m above graft union; length 150 cm; diameter 7.0 cm; crotch angle 70′; color light brown RHS N200D.
Dormant one year old shoot:
   Size.—Diameter 4-5 mm; Length 20-30 cm.
   Color.—Brown RHS 200C.
   Internode length.—2.0 cm.
   Pubescence.—Very strong.
   Lenticels.—Size 1-2 mm, density 1-2/cm².
Flowers:
   Bud.—Quantity per spur 4 to 5; length 8-10 mm; shape globose; color red-purple RHS 63A.
   Petals.—Quantity per flower 5; Margins free to touching; Apex shape round; Base shape round; Length 16-18 mm; Width 15-16 mm; upper surface color white RHS 155D; Lower surface color white RHS 155D with traces of pink RHS 68B in veins and edges when fully open.
   Flower.—Diameter of fully open flower 2 cm; Quantity per cluster 4.
   Sepals.—Quantity per flower 5; Shape triangular; color green RHS 135B.
   Pedicel.—Length 19-21 mm; Diameter 2 mm; Color greyed-purple RHS 187A.
   Pistil.—Length 13-14 mm; Color yellow-green RHS 145C.
   Anthers.—Quantity per flower 16; length 2 mm; pollen color yellow RHS 12B.
   Stigma.—Size 1 mm; Color RHS 11C.
   Style.—Length 7 mm; color yellow-green RHS 145C.
   Bloom period.—Early to Mid Season; first bloom April 8, full bloom April 12 at Hérault, France.
Leaf:
   Attitude in relation to shoot.—Outwards.
   Size.—Length 10-11 cm; Width 5.0-6.0 cm; Length to Width Ratio 1.8-2.0.
   Margin.—Serrate.
   Color.—Upper surface yellow-green RHS 146C; lower surface yellow-green RHS 146C.
   Shape.—Ovate; apex acuminate; base aequilateral.
   Petiole.—Length 1.5-2.0 cm; width 2 mm; color yellow-green RHS 144C.
Fruit:
   Size.—Weight 150 g; diameter 75-90 mm.
   Fruit shape.—Cylindrical.
   Position of maximum diameter.—Top.
   Ribbing.—Absent.
   Aperture of eye.—Closed.
   Depth of eye basin.—8-10 mm.
   Width of eye basin.—35-40 mm.
   Stalk.—Diameter 3 mm; length 18-22 mm; Color RHS 199B.
   Depth of stalk cavity.—11-13 mm.
   Width of stalk cavity.—20 mm.
   Lenticels.—Size 0.6-0.8 mm; density 9-11/cm².
   Bloom of skin.—Absent.
   Greasiness of skin.—Absent.
   Ground color of skin.—Yellow-green RHS 154B.
   Over color of skin.—Pink-Red RHS 47C.
   Amount of over color.—50-80 percent.
   Pattern of over color.—Solid flush with weakly defined stripes.
   Brix.—13-15.
   Flesh.—Texture juicy, firm, about 9.5 kg/cm²; color white RHS 155C.
   Seeds.—Quantity per fruit 6-8; teardrop shape; color brown RHS 200B.
   Aroma.—Strong.
   Harvest date.—Medium-late August.
Resistance/susceptibility to known diseases/pests: Susceptible to grey aphids.

1. We claim a new and distinct apple tree substantially as shown and described herein.
